(19) United States
(12) Patent Application Publication
(10) Pub. No.: US 2002/0068724 A1
(43) Pub. Date: Jun. 6, 2002

(54) Method and composition of novel compounds for the therapy and targeting of the primary modalities of cancer cell proliferation and homeostasis

(75) Inventors: Thomas J. Slaga, Austin, TX (US); Addanki P. Kumar, Denver, CO (US); William Alworth, New Orleans, LA (US)

Correspondence Address: DAVID G. HENRY, 900 Washington Avenue, P. O. Box 1470, Waco, TX, 76701, US

(73) Assignee: Oncology Sciences Corporation, Austin, TX (US)

(21) Appl. No.: 09/777,151

(22) Filed: Feb. 5, 2001

Related U.S. Application Data
(63) Continuation-in-part of application No. 09/527,283, filed on Mar. 17, 2000, now abandoned.

(51) Int. Cl. A61K 31/56

(52) U.S. Cl. 514/169; 514/182

(57) ABSTRACT

The invention is of both a composition and method for inhibiting the proliferation of cancerous cells. The composition is, and the method is based on the use of a composition consisting (among active ingredients) substantially of 2-methoxyestradiol and/or one of a number of anaologues thereof. The present inventors have demonstrated beyond serious doubt that these compounds have a pronounced effect in inhibiting the proliferation of cancerous cells and, therefore, provide a desperately needed stepping stone for advancing toward meaningful treatment of cancer.

### CITATION TO PRIOR APPLICATION

This is a continuation-in-part with respect to U.S. application Ser. No. 09/527283, filed Mar. 17, 2000 from which priority is claimed under 35 U.S.C. §120 and under provisions of the Patent Cooperation Treaty.

### BACKGROUND OF THE INVENTION

A. FIELD OF THE INVENTION

The present invention concerns novel chemical compounds, the chemical synthesis of said novel chemical compounds, and the use of said compounds in the treatment of a broad array of cancers.

B. BACKGOUND OF THE INVENTION

1. The Problem: Primary Modalities of Cancer Cell Growth and Expansion

Cancer is the second leading cause of death in the United States, accounting for approximately one in four deaths. Recent estimates by the American Cancer Society suggest that in excess of 500,000 people die from cancer every year—that is approximately 1,500 deaths a day. Further, approximately 2.5 million new cases of cancer were expected to be diagnosed in the year 2000 alone. At an estimated annual cost of $107 billion dollars in health care costs and lost productivity due to death and illness, cancer inflicts a vast human and monetary toll on the United States.

The generic use of the term “cancer” only hints at the vast diversity of anatomical structures that this disease affects and the myriad of molecular bases that form the foundation of this disease. The collective use of the word cancer includes diseases affecting the brain, breast, cervix uteri, colon, corpus uteri, kidney, renal pelvis, larynx, lung, bone marrow, bronchus, skin, lymph system, nervous system, oral cavity, pharynx, ovary, pancreas, prostate, rectum, stomach, testis, thyroid, urinary bladder, and others. The individual molecular bases of these diverse afflictions can be varied and diverse. However, among this diverse field of afflictions, there exist two unified modalities of cell growth and/or proliferation that are common to almost all types of cancer: 1) unchecked cell growth and/or immortality, and 2) angiogenesis.

On of the problems that characterize a vast number of cancers is the unregulated growth or unchecked life span of aberrant cells in the various tissues of the body. Normal cells grow, divide, and die on a regular basis. The process by which cells normally die is called apoptosis. However, when normal cell growth and death become unchecked in the body, by any number of processes, such unchecked growth and/or immortality leads to the formation of cancerous tumors or cell populations that can interfere and ultimately destroy the regular functioning of the various tissues of the body. Such growth or immortality can ultimately lead to the occurrence of a host of solid tumors, leukemia's, lymphomas, or the metastasis of cancer cells throughout the body. Unchecked cell growth and/or immortality are problematic biological mechanisms common to almost all types of cancer.

Another biological mechanism that is common to, and problematic in the treatment of, all solid cancer tumors is angiogenesis. Angiogenesis refers to the process by which new blood vessels are formed in the body. Without a dedicated blood supply, solid tumors have only limited growth potential—perhaps 2 mm in diameter maximum. However, angiogenesis often occurs in cancerous tissues and tumors, thus enabling solid tumors to sequester greater amounts of nutrients from the body and allowing them to proliferate rapidly, even spreading to other parts of the body. Angiogenesis is a critical means by which solid tumors grow rapidly and metastasize, hastening the process of death or disfigurement.

These two independent biological mechanisms are the common, primary modalities by which almost all cancer cells proliferate and grow. Hence, a novel approach for the treatment of cancer would be the development of pharmacological agents that have dual roles as anti-angiogenic as well as pro-apoptotic agents. Such an agent will have the ability to target both components of a cancer: kill the tumor cell by induction of apoptosis and cut off the blood supply to the tumor cell so that it will not grow.

Therefore, there exists an urgently compelling, yet unsatisfied need to develop strategies for the development of a class of compounds that have both anti-angiogenic as well as pro-apoptotic properties.

2. One Solution: Analogues of 2-methoxyestradiol (2-ME)

A recent breakthrough in the treatment of cancer is the use of 2-methoxyoestradiol (hereinafter “2-ME”). 2-ME is an endogenous non-toxic metabolic byproduct of estrogens that is present in human urine and blood. (1) A potential role for 2-ME as a chemopreventive agent has been reported in the mammary and pancreatic models. (2) 2-ME has also been shown to inhibit endothelial cell proliferation implicating its potential role in angiogenesis. (3) In addition, apoptosis has been implicated as a mechanism for 2-ME's cytostatic and anti-angiogenic effect. The present inventors previous work, filed with the original patent application and another continuation in part, shows that 2-ME is of great significance in the treatment of prostate, brain, and nervous system cancer through the induction of apoptosis. This body of work indicates that 2-ME is an antitumorigenic agent with a significant therapeutic advantage since it can preferentially inhibit actively proliferating cells (characteristic of tumor cells) without affecting the growth of normal cycling cells. Additionally, 2-ME appears to also inhibit the formation of new blood vessels. To the best of our knowledge, this is the first compound that targets two components of cancer: the tumor cells and their blood supply. The present inventors have demonstrated that 2-ME is a chemical compound with a significant role as an antitumorigenic agent with broad efficacy in a variety of cancerous cell populations.

Building on these findings, further experiments have helped to elucidate the structural bases for 2-ME's molecular efficacy. A number of experiments have been conducted using 2-ME and 16-epiestriol (hereinafter “16-ES”), an analogue of 2-ME that lacks the methoxy group at the second position. In a multitude of experiments, using prostate cancer cell lines (both androgen-dependent (LNCaP), and androgen-independent (DU145) cells), and a brain and/or nervous system cancer cell line (DAOY), the present inventors have studied the effects of 2-ME and 16-ES on cell proliferation and the induction of apoptosis, in a number of ways. The sum of all the data clearly indicates that 2-ME is a compound that significantly inhibits cancerous cell growth and has proapoptotic effects, while 16-ES does not. In total, these data suggests that the efficacy of 2-ME may be associated with the methoxy moiety at the second position of 17p-estradiol (E₂). Further, it also suggests the possible efficacy of a series of compounds with various moieties at the second position in the treatment of cancer. Additionally, the specific anti-proliferative, pro-apoptotic, anti-angiogenesis, and other efficacy of 2-ME against cancer cells suggests that other structural modifications of the molecule should be explored in attempts to increase the efficacy of the agent. Thus, the present inventors now propose a method of synthesizing a number of analogues of 2-ME that may possess enhanced efficacy in the treatment of cancer. These analogues are prepared as described herein and are designed (1) to determine which components of the 2-ME molecule in addition to the 2-methoxy group are required for the observed chemopreventive effects and (2) to determine if other useful 2-ME analogues can be created that are effective in the treatment of cancer or other diseases.

### SUMMARY OF THE INVENTION

It is an object of the present invention to provide an agent or composition, or more than one agent or composition, that is efficacious in inhibiting the proliferation and/or angiogenesis of cancer cells.

It is another object of the present invention to provide a method for creating novel molecules that are efficacious in inhibiting the proliferation and/or angiogenesis of cancer cells.

It is another object of the present invention to provide a composition the primary active ingredient of which are an analogue or analogues of 2-methoxyestradiol which are efficacious in inhibiting the proliferation and/or angiogenesis of cancer cells.

It is another object of the present invention to provide a method for inhibiting the proliferation and/or angiogenesis of cancer cells through use of a composition the primary active ingredient of which is 2-methoxyestradiol or an analogue thereof, as described herein.

In satisfaction of these and related objectives, the present invention provides both a method and composition for inhibiting the proliferation of cancerous cells. The method is, and the composition is based on the use of a composition consisting (among active ingredients) substantially of 2-methoxyestradiol and/or one of a number of analogues thereof. The present inventors have demonstrated beyond serious doubt that these compounds may have a pronounced effect in inhibiting the proliferation of cancerous cells and, therefore, provide a desperately needed stepping stone for advancing toward meaningful treatment of cancer.

### DETAILED DESCRIPTION OF THE PREFERED EMBODIMENT

Data from the present inventors laboratory shows that 2-ME inhibits the growth of brain, nervous system and prostate cancer cells but that 16-epiestriol does not. This indicates that substituting the second position of 17b-estradiol (E₂) with a methoxy group generates a molecular structure that shows significant and selective growth inhibitory activity toward prostate cancer cells while simultaneously eliminating the potentially detrimental growth stimulating activity of E₂ itself. The analogues of 2-ME to be prepared as described below are designed (1) to determine which components of the 2-ME molecule in addition to the 2-methoxy group are required for the observed chemopreventive effects and (2) to determine if growth-inhibitory 2-ME analogues can be created that are effective.

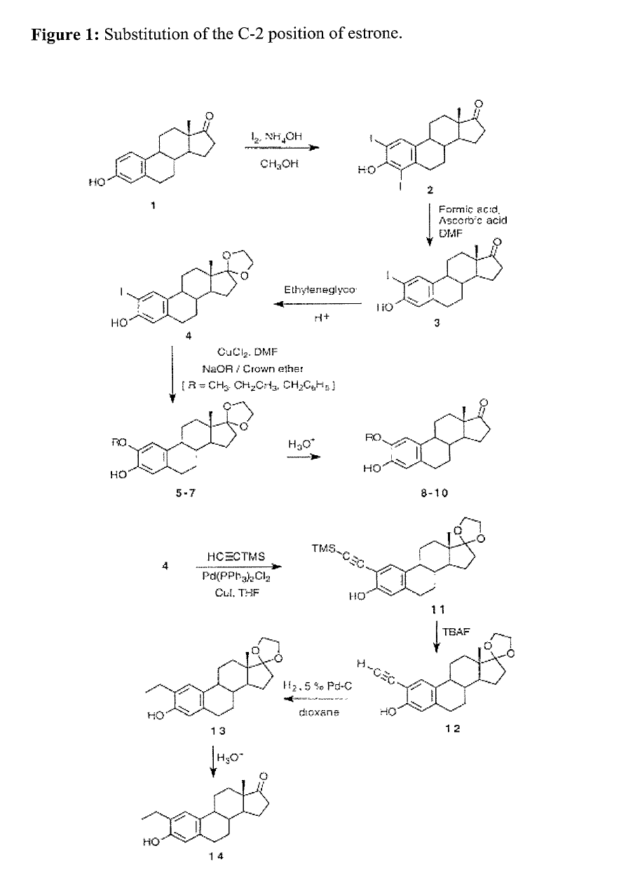

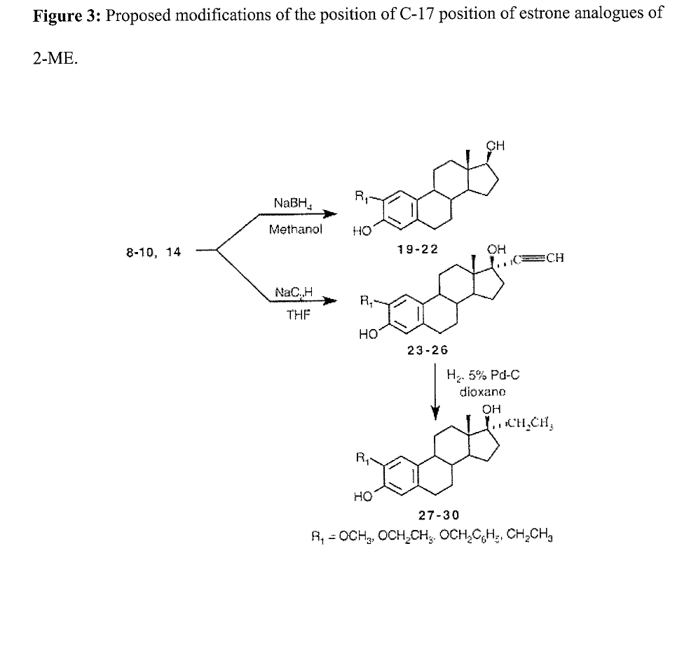

The initial compounds to be synthesized will be 2 alkoxy substituted analogues of estrone shown in FIG. 1. These compounds will then be converted into the 2-ME analogues as shown in FIG. 3 (analogues 19-21, 23-25, and 27-29).

FIG. 1 illustrates how the A ring of the E₂ steroidal nucleus will be modified to generate 2-alkoxy substituted analogues of estrone (analogues 8-10) and a 2-ethyl substituted estrone analogue (analogue 14). The key reactions in this figure are the synthesis of compound 2,2,4-diiodoestrone, and its conversion to compound 3, the 2-iodoestrone derivative. The iodination and diodination of the estrone starting material (analogue 1) will be carried out as described by Ikegawa et al in their synthesis of catecholic equilin and equilin derivatives. (4) The proposed conversion of the ethylenedioxy protected 2-iodoestrone derivative 4 to the protected 2-methoxy, 2-ethoxy, and 2 benzyloxy derivatives 5-7 by Cu (I) catalyzed reactions of the alkoxides in dimethylformamide in the presence of a crown ether is based upon the comparable reaction of a protected 2-iodoequilin also described by Ikegawa et. al in the synthesis of catechol equilins. (4) It should be noted that if it proves necessary the estrone starting material used in FIG. 1 could be protected as the ethylenedioxy derivative by treatment with ethylene glycol prior to the iodination reaction. The Pd(Ph₃)Cl₂/CuI catalyzed coupling of the aryl iodide (analogue 4) with trimethylsilyl substituted acetylene to yield the 2-alkynyl substituted estrone derivative 11 shown in FIG. 1 has many known precedents (5). The present inventors have carried out many such coupling reactions in their laboratory and have found that molecules containing active hydrogens (NH₂ or OH groups) can be successfully coupled in such reactions if care is taken to form the reactive Cu-TMS acetylene complex before the halogenated aromatic substrate is added. It is therefore anticipated that this reaction will proceed as shown in FIG. 1. If, however, the reaction fails to be successful as shown in FIG. 1, the intermediate 4 will be coupled with trimethylsilylacetylene in 9:1 CH₃CN/H₂O catalyzed with Pd(AcO)₂/PPh₃/CuI. The present inventors have carried out a model reaction in their laboratory with an unprotected iodophenol that gave the desired coupling product with this procedure.

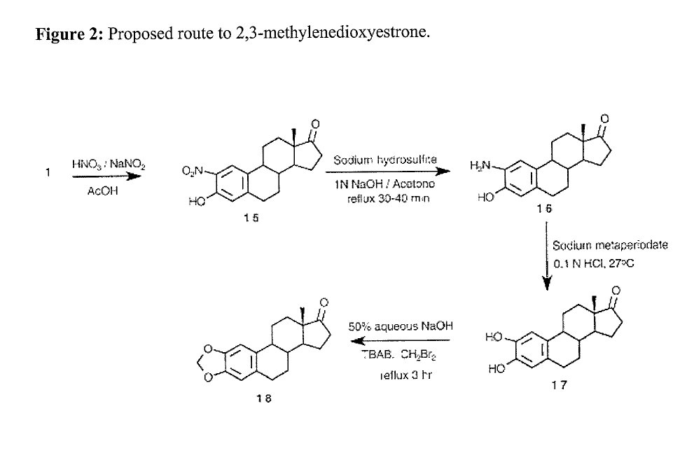

FIG. 2 outlines the reaction sequence that will be employed to prepare the 2,3-methylenedioxyestrone derivative (analogue 18). This reaction sequence is based upon the reaction sequence employed by Stubenrauch and Knuppen to prepare catechol estrogens. (6)

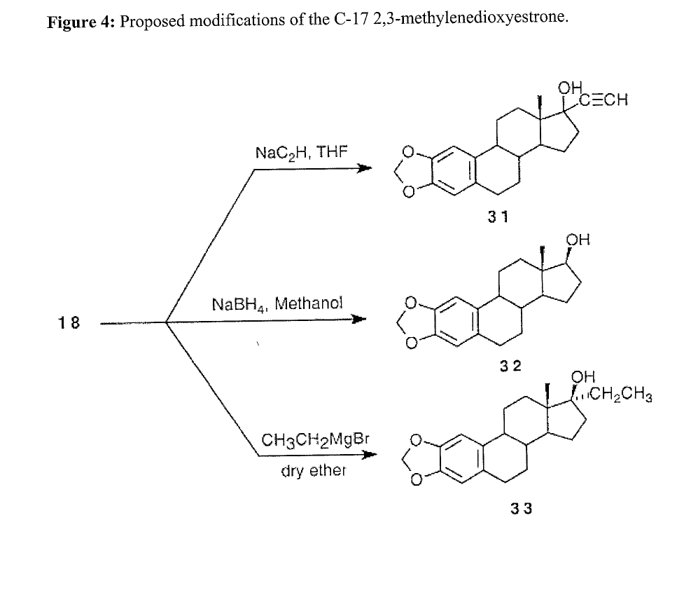

FIGS. 3 and 4 illustrate how 2-methoxyestrone and the 2-methoxyestrone analogues prepared as outlined in FIGS. 1 and 2 above will be converted into (i) 2-methoxyestrone and its analogues and (ii) 2,3-methylenedioxyestrone analogues modified at position C-17. The preparation of these structures will not only allow us to test the requirement for the 17b-hydroxyl group in the chemopreventive activity of 2-ME but will also enable us to determine if substitutions at C-17 (for example, the 17-ethynyl2-ME derivative, 23) will decrease the rate of metabolism and deactivation of 2-ME and its analogues. As outlined in FIGS. 3 and 4 below, the present inventors propose to prepare both 2-ethyl-17b-estradiol (analogue 22) and 2,3-methylenedioxy-17b-estradiol (analogue 32). In addition, since 17a-ethynylestradiol (ethynylestradiol) is both a potent estrogenic and long-lived analogue of E₂, the 17a-ethynyl derivative of 2-ME (analogue 19) will be prepared as outlined in FIG. 3. In addition, by directing synthesis to produce estrone analogues of the target structures (analogues 8-10, 14, and 18) as illustrated in FIGS. 1 and 2, it will be possible to prepare 17a-ethynyl, and 17a-ethyl derivatives of the 2-alkoxy, 2-ethyl, and 2,3-methylenedioxy analogues (analogues 23-26, 27-30, 31 and 32).

It should be noted that the proposed reactions used to modify the C-17 carbonyl of the estrone analogues shown in FIGS. 3 and 4 are standard reactions that have been successfully applied to estrone. (7)

Although not explicitly shown in FIG. 1 and 3, the 2-ethynyl intermediate shown in FIG. 1 (analogue 12) will also be converted into 2-ethynylestrone and 2-ethynylestradiol for testing. Further, although not explicitly indicated in FIGS. 1 and 2, the 2-ethynylestrone derivative 11 shown in FIG. 1 will also be converted into 2-ethynylestrone and 2-ethynylestradiol as shown in FIG. 2 for the other intermediates. This will generate two additional 2-ME analogues for biological testing. Lastly, it is also possible to modify the acetylene coupling reaction shown in FIG. 1 to prepare 2-(1-propynyl) and 2-(1-butynyl) derivatives of 2-ME that could serve as precursors of 2-propyl and 2-butyl 2-ME analogues.

The synthesis reactions in FIGS. 1-4 outlined above will provide an efficient way of generating 2-ME (analogue 19) and fourteen 2-ME analogues (analogues 20-33) that can be utilized to determine the effects of modifying both the C-17 and the C-2 position of 2-ME. Samples of the estrone analogues themselves (analogues 8-10, 14, 18) will also be tested for their potential growth-inhibitory activity. The reaction sequences outlined in FIGS. 1-4 will therefore produce a total of 21 new 2-ME analogues to be tested as potential selective inhibitors of cancer cell growth and angiogenesis. It is anticipated that one or more of these analogues may manifest selective growth-inhibitory activities towards cancer cells while, at the same time, being less subject to metabolic conversions that will deactivate or eliminate these active analogues. It is also likely that 17a-ethylyl derivative of 2-ME may have a longer effective half-life both in vitro and in vivo.

### References

1. Gelbke, H. P., and Knuppen, R. 1976. The exertion of five different 2-hydroxyestrogen monomethyl ethers in human pregnancy urine. J Steroid Biochem. 7: 457-463.

2. Zhu, B. T. and Conney, A. H. 1998. Is 2-methoxyestradiol an endogenous estrogen metabolite that inhibits mammary carcinogenesis. Cancer Res. 58: 2269-2277.

3. Fotsis, T., Zhang, Y., Pepper, M. S., Adlercreutz, H., Montesano, R., Nawroth, P. P. and Schweigerer, OL. 1994. The endogenous estrogen metabolite 2-methoxyestradiol inhibits angiogenesis and suppresses tumor growth. Nature. 368: 237-239.

4. Ikegawa, S., Kurosawa, T., and Tohma, M. (1988) Syntheses of C-2 caecholic equilin and equilin derivatives for use in metabolic studies. Chem. Pharm Bull. 36:2993-2999.

5. Neenan, T. X., and Whitesides, G. M. (1988) Synthesis of high carbon monomers bearing multiple ethynyl groups. J. Org. Chem., 53:2489-2496.

6. Stubenrauch, G. And Knuppen, R. (1976) Convenient large scale preparation of catechol estrogens. Steroids, 28:733-741.

7. Fieser, L. F. And Fieser, M. (1959) Estrogens in Steroids, Chapter 15, 444-502, Chapman and Hall, Ltd. London.

We claim:

1. The use of compositions useful in the inhibition of cancerous cell proliferation selected from a group consisting of:
the 2-ethyl-17-β-estradiol molecules identified as analogues 20-22 in FIG. 3, specifically excluding any claim to 2-methyloxyestradiol;
the 17-α-ethynyl molecules identified as analogues 23-26 in FIG. 3;
the 17-α-ethyl molecules identified as analogues 27-30 in FIG. 3;
the 2,3-methylenedioxy molecules identified as analogues 31, 32, and 33 in FIG. 4;
the 2-alkoxy substituted analogues of estrone molecules identified as analogues 8-10 in FIG. 1;
the 2-ethyl substituted molecule identified as analogue 14 in FIG. 1; and
the 2,3-methylenedioxyestrone molecule identified as analogue 18 in FIG. 2.

2. A method for inhibiting cancerous cell proliferation comprising the steps of:
selecting a composition from the group consisting of the 2-ethyl-17-β-estradiol molecules identified as analogues 20-22 in FIG. 3, specifically excluding any claim to 2-methyloxyestradiol, the 17-α-ethynyl molecules identified as analogues 23-26 in FIG. 3, the 17-α-ethyl molecules identified as analogues 27-30 in FIG. 3, the 2,3-methylenedioxy molecules identified as analogues 31, 32, and 33 in FIG. 4, the 2-alkoxy substituted analogues of estrone molecules identified as analogues 8-10 in FIG. 1, the 2-ethyl substituted molecule identified as analogue 14 in FIG. 1, or the 2,3-methylenedioxyestrone molecule identified as analogue 18 in FIG. 2; and
administering said composition to cells in which is identified suspected cancer cells.

3. The method of claim 2 wherein said suspected cancer cells are brain cancer cells.

4. The method of claim 2 wherein said suspected cancer cells are nervous system cancer cells.

5. The method of claim 2 wherein said suspected cancer cells are brain cancer cells and nervous system cancer cells.

6. The method of claim 2 wherein said suspected cancer cells are prostate cancer cells.

7. A composition for application to cancerous cells consisting in active constituents substantially of one or more agents chosen from the 2-ethyl-17-β-estradiol molecules identified as analogues 20-22 in FIG. 3, specifically excluding any claim to 2-methyloxyestradiol, the 17-α-ethynyl molecules identified as analogues 23-26 in FIG. 3, the 17-α-ethyl molecules identified as analogues 27-30 in FIG. 3, the 2,3-methylenedioxy molecules identified as analogues 31, 32, and 33 in FIG. 4, the 2-alkoxy substituted analogues of estrone molecules identified as analogues 8-10 in FIG. 1, the 2-ethyl substituted molecule identified as analogue 14 in FIG. 1, or the 2,3-methylenedioxyestrone molecule identified as analogue 18 in FIG. 2.

8. The method of claim 8 wherein said suspected cancer cells are brain cancer cells.

10. The method of claim 8 wherein said suspected cancer cells are nervous system cancer cells.

11. The method of claim 8 wherein said suspected cancer cells are brain cancer cells and nervous system cancer cells.

12. The method of claim 8 wherein said suspected cancer cells are prostate cancer cells.
